(19) United States
(12) Patent Application Publication
(10) Pub. No.: US 2025/0009313 A1
(43) Pub. Date: Jan. 9, 2025

(54) VARIABLE THRESHOLDS FOR AUTOREGULATION

(71) Applicant: Covidien LP, Mansfield, MA (US)

(72) Inventors: Paul S. Addison, Edinburgh (GB); Dean Montgomery, Edinburgh (GB)

(21) Appl. No.: 18/739,455

(22) Filed: Jun. 11, 2024

Related U.S. Application Data
(60) Provisional application No. 63/511,796, filed on Jul. 3, 2023.

(51) Int. Cl. A61B 5/00; A61B 5/021; A61B 90/92; G06F 3/04847; G06F 3/0488

(52) U.S. Cl. CPC A61B 5/743; A61B 5/021; A61B 90/92; G06F 3/04847; G06F 3/0488; A61B 2505/05

(57) ABSTRACT

An autoregulation monitoring device may display, on a graph on the display screen, a blood pressure signal of a patient; indicate, on the graph, an autoregulation threshold that separates an intact autoregulation area of the graph and an impaired autoregulation area of the graph. The device may indicate, on the graph, a plurality of threshold offsets that are each separated from the autoregulation threshold, wherein each one of the plurality of threshold offsets is selectable by a user. The device may receive an indication of user input that corresponds to selection of a threshold offset from the plurality of threshold offsets and may update the autoregulation threshold on the graph based on the selected threshold offset.

### CROSS-REFERENCE TO RELATED APPLICATION

The present application claims the benefit of U.S. Provisional Patent Application Ser. No. 63/511,796, filed on Jul. 3, 2023, the entire content of which is incorporated herein by reference.

### TECHNICAL FIELD

This disclosure relates to monitoring autoregulation status of a patient.

### BACKGROUND

Clinicians may monitor one or more physiological parameters of a patient, e.g., to monitor a patient's autoregulation status. Autoregulation is the response mechanism by which an organism regulates blood flow over a wide range of systemic blood pressure changes through complex myogenic, neurogenic, and metabolic mechanisms. During autoregulation, arterioles dilate or constrict in an attempt to maintain appropriate blood flow. Autoregulation may occur for a variety of organs and organ systems, such as, for example, the brain, the kidneys, the gastrointestinal tract, and the like. In the example of cerebral autoregulation, as cerebral blood pressure decreases, cerebral arterioles dilate in an attempt to maintain blood flow. As cerebral pressure increases, cerebral arterioles constrict to reduce the blood flow that could cause injury to the brain.

### SUMMARY

The present disclosure describes example devices, systems, and techniques for displaying information related to an autoregulation status of a patient, which may include the cerebral autoregulation status of the patient and/or one or more non-cerebral autoregulation statuses of the patient. The devices and systems described herein may display information related to the autoregulation status of a patient by displaying a graph of a blood pressure value associated with the patient over time against an autoregulation threshold, such as a lower limit of autoregulation (LLA). The autoregulation threshold may separate an intact autoregulation region in the graph from an impaired autoregulation region. A clinician may view the graph of the blood pressure value associated with the patient to determine whether the blood pressure value associated with the patient is in the intact autoregulation region or the impaired autoregulation region to determine the autoregulation status of the patient.

In some aspects, the techniques described herein relate to a method for displaying autoregulation status including: displaying, on a graph on a display screen, a blood pressure signal of a patient; indicating, on the graph, an autoregulation threshold that separates an intact autoregulation area of the graph and an impaired autoregulation area of the graph; indicating, on the graph, a plurality of threshold offsets that are each separated from the autoregulation threshold, wherein each one of the plurality of threshold offsets is selectable by a user; receiving, by one or more processors, an indication of user input that corresponds to selection of a threshold offset from the plurality of threshold offsets; and updating, by the one or more processors, the autoregulation threshold on the graph based on the selected threshold offset.

In some aspects, the techniques described herein relate to a device including: memory; and processing circuitry configured to: display, on a graph on the display screen, a blood pressure signal of a patient; indicate, on the graph, an autoregulation threshold that separates an intact autoregulation area of the graph and an impaired autoregulation area of the graph; indicate, on the graph, a plurality of threshold offsets that are each separated from the autoregulation threshold, wherein each one of the plurality of threshold offsets is selectable by a user; receive an indication of user input that corresponds to selection of a threshold offset from the plurality of threshold offsets; and update the autoregulation threshold on the graph based on the selected threshold offset.

In some aspects, the techniques described herein relate to a non-transitory computer readable medium including instructions that, when executed by processing circuitry, cause the processing circuitry to: display, on a graph on the display screen, a blood pressure signal of a patient; indicate, on the graph, an autoregulation threshold that separates an intact autoregulation area of the graph and an impaired autoregulation area of the graph; indicate, on the graph, a plurality of threshold offsets that are each separated from the autoregulation threshold, wherein each one of the plurality of threshold offsets is selectable by a user; receive an indication of user input that corresponds to selection of a threshold offset from the plurality of threshold offsets; and update the autoregulation threshold on the graph based on the selected threshold offset.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual block diagram illustrating an example system configured to monitor an autoregulation status of a patient.

FIG. 2 is a conceptual block diagram illustrating an example autoregulation monitoring device.

FIGS. 3A-3H illustrate example graphical user interfaces outputted by an example autoregulation monitoring device.

FIG. 4 is a flow diagram illustrating an example technique for presenting the autoregulation status of a patient.

### DETAILED DESCRIPTION

In general, aspects of this disclosure describe devices, systems, and techniques that may determine an autoregulation status of a patient, such as a cerebral autoregulation status of a patient and/or or more non-cerebral autoregulation status of the patient, and may present the autoregulation status of the patient in ways that enable a clinician to make finer-grained determinations of the autoregulation status of the patient. An intact autoregulation status of a subject occurs over a range of blood pressures defined between a lower limit of autoregulation (“LLA”) and an upper limit of autoregulation (“ULA”). An impaired autoregulation status occurs outside of the range of blood pressures defined between the LLA and the ULA and may occur when a patient's autoregulation process is not functioning properly. When a patient exhibits an impaired autoregulation status, the patient may experience inappropriate blood flow, which may be undesirable. For example, below a respective LLA, a drop in blood flow to a respective organ may cause ischemia and adversely affect the respective organ. Above a respective ULA, an increase in blood flow to a respective organ may cause hyperemia, which may result in swelling or edema of the respective organ. A clinician may monitor the autoregulation status of a patient, e.g., during a medical procedure, and take one or more actions to keep the patient in or bring the patient to an intact autoregulation status, such as by increasing or decreasing the patient's blood pressure.

A device configured to monitor an autoregulation status of a patient may be configured to determine an autoregulation status value based on various physiological parameters of the patient, such as a blood pressure signal indicative of a blood pressure of a patient and an oxygen saturation signal indicative of blood oxygen saturation (e.g., regional oxygen saturation) of a patient. For example, an autoregulation monitoring device may include processing circuitry configured to receive a blood pressure signal indicative of a blood pressure of the patient and an oxygen saturation signal indicative of an oxygen saturation of the patient. The blood pressure of the patient may be obtained by any suitable blood pressure measurement technique. In some examples, the blood pressure of the patient may include an arterial blood pressure measured using a non-invasive blood pressure measurement, such as a blood pressure derived from external cuff or photoplethysmogram, or an invasive blood pressure, such as a blood pressure derived from an intra-arterial blood pressure monitor. For example, the blood pressure value may include, or be representative of, the middle cerebral artery in the brain of the patient. The oxygen saturation of the patient may include an oxygen saturation value for any suitable patient anatomy. For example, the oxygen saturation value may include, or be representative of, an oxygen saturation at the brain of the patient.

The processing circuitry may determine a metric (e.g., a numerical value or qualitative information) indicative of the cerebral autoregulation status of the patient based on the blood pressure signal and the oxygen saturation signal. For example, a cerebral autoregulation status value may include a limit of cerebral autoregulation, such as LLA and/or ULA, of the patient that may be determined based on the blood pressure signal and the oxygen saturation signal. In some examples, the LLA and/or the ULA may be determined based on cerebral perfusion pressure. Cerebral perfusion pressure may be determined based on the blood pressure signal and intracranial pressure of the patient. In some examples, the processing circuitry may determine the LLA and/or the ULA based on a correlation index (COx) of the blood pressure value and oxygen saturation value. Alternatively or additionally, the processing circuitry may determine the LLA and/or the ULA based on other parameters or correlation coefficients.

In some examples, the processing circuitry may determine the LLA and/or the ULA based on a comparison of a threshold value to a change in the blood pressure (and/or oxygen saturation) of a patient over time, e.g., determining a correlation coefficient only if the change in blood pressure (and/or oxygen saturation) over time exceeds the threshold value. In some examples, as described in commonly assigned U.S. Patent Application Publication No. 2018/0014791 naming inventors Montgomery et al. and entitled, “SYSTEMS AND METHODS OF MONITORING AUTOREGULATION,” which is hereby incorporated by reference in its entirety, the processing circuitry may process a blood pressure signal and an oxygen saturation signal to determine respective gradients of the signals (i.e., a blood pressure gradient and an oxygen saturation gradient) over a period of time and determine the patient's autoregulation status based on the respective gradients. As described in U.S. Patent Application Publication No. 2018/0014791, the processing circuitry may determine the autoregulation system of the patient may be impaired if the blood pressure gradient and the oxygen saturation gradient trend together (e.g., change in the same direction) over a period of time. In some cases, the processing circuitry may determine that the autoregulation system of the patient may be intact if the blood pressure gradient and the oxygen saturation gradient do not trend together (e.g., do not change in the same direction, such as change in different directions, or the blood pressure changes while the oxygen saturation remains generally stable) over the period of time.

Other systems and techniques using similar or different parameters may be used to determine a limit of cerebral autoregulation. For example, as described in commonly assigned U.S. Patent Application Publication No. 2017/0105631 filed on Oct. 18, 2016, entitled “Systems and Method for Providing Blood Pressure Safe Zone Indication During Autoregulation Monitoring,” and U.S. Patent Application Publication No. 2017/0105631 filed on Oct. 18, 2016, entitled “System and Method for Providing Blood Pressure Safe Zone Indication During Autoregulation Monitoring,” systems and methods for monitoring autoregulation may use an autoregulation index to generate and display an autoregulation profile (e.g., autoregulation index values sorted into bins corresponding to different blood pressure ranges) of the patient, and generate a blood pressure (BP) safe zone (e.g., designate a blood pressure range encompassing one or more of the bins) indicative of intact autoregulation. As another example, as described in commonly assigned U.S. Patent Application Publication No. 2016/0367197 filed on Jun. 6, 2016, entitled “Systems and Methods for Reducing Signal Noise When Monitoring Autoregulation,” systems and methods for monitoring autoregulation may determine linear correlations between measured physiological parameters using regression analyses, such as a least median of squares (LMS) regression method or a least trimmed squares regression method (LTS), applied to oxygen saturation measurements plotted against blood pressure measurements to determine a regression line associated with COx to ignore or exclude data outliers representative of the noise, and to utilize the remaining data to determine the COx or HVx.

The processing circuitry may provide a signal indicative of the cerebral autoregulation status value and/or the non-cerebral autoregulation status value to an output device. For example, the processing circuitry may output a graphical user interface (GUI) that is displayed by a display device that enables a clinician to monitor the autoregulation status of the patient, such as during surgery. The clinician may monitor the autoregulation status of the patient to correct an impaired autoregulation status to reduce adverse effect to the brain, an organ, or organ systems of the patient. For example, during surgery an anesthesiologist may adjust therapy based on the information indicative of the autoregulation status to improve the autoregulation statuses, e.g., to preserve intact status. Providing information indicative of the autoregulation status may better help the clinician avoid organ dysfunction that might otherwise occur.

An autoregulation monitoring device that determines the LLA of patients, such as by using any of the techniques, may not always be able to accurately determine an LLA of a patient that is undergoing a medical procedure. For example, the patient may have a particular medical condition that causes the patient to have an LLA that is different from the LLA determined by the autoregulation monitoring device, or the patient may be undergoing a medical procedure for which the autoregulation monitoring device may be unable to accurately determine the LLA of the patient. Furthermore, new medical studies may be published after the autoregulation monitoring device has been put in use that may indicate that it may be optimal to keep the blood pressure value of a patient above a blood pressure level that is different from the LLA of the patient. As such, if the processing circuitry outputs the autoregulation status of the patient that merely indicates whether the blood pressure value of the patient is above or below the LLA determined by the autoregulation monitoring device, the autoregulation monitoring device may present an incorrect or less than optimal autoregulation status of the patient.

In accordance with aspects of this disclosure, the processing circuitry of an autoregulation monitoring device may output a GUI that is displayed by a display device that enables a clinician to monitor the autoregulation status of the patient. The GUI may include a graph of a blood pressure value of the patient over time, and may include, in the graph an autoregulation threshold that separates an intact autoregulation region from an impaired autoregulation region. The autoregulation threshold may be set to the LLA of the patient or may be set to a value that is different from the LLA, such as by being set to the LLA of the patient plus or minus an offset value. A clinician may monitor the GUI to determine the autoregulation status of the patient based on whether the blood pressure value of the patient is in the intact autoregulation region or the impaired autoregulation region.

The graph also includes autoregulation threshold offsets that are offset from each other and from the autoregulation threshold by a specified blood pressure offset. Thus, if the clinician knows that the autoregulation threshold presented in the GUI is not an accurate representation of the LLA of the patient, the clinician may instead monitor the blood pressure value of the patient against a particular autoregulation threshold offset and may monitor the blood pressure value in the GUI against the particular autoregulation threshold offset. In this way, the autoregulation monitoring device may enable a clinician to more accurately determine the autoregulation status of a patient even in situations where the autoregulation monitoring device is unable to accurately determine the LLA of a patient.

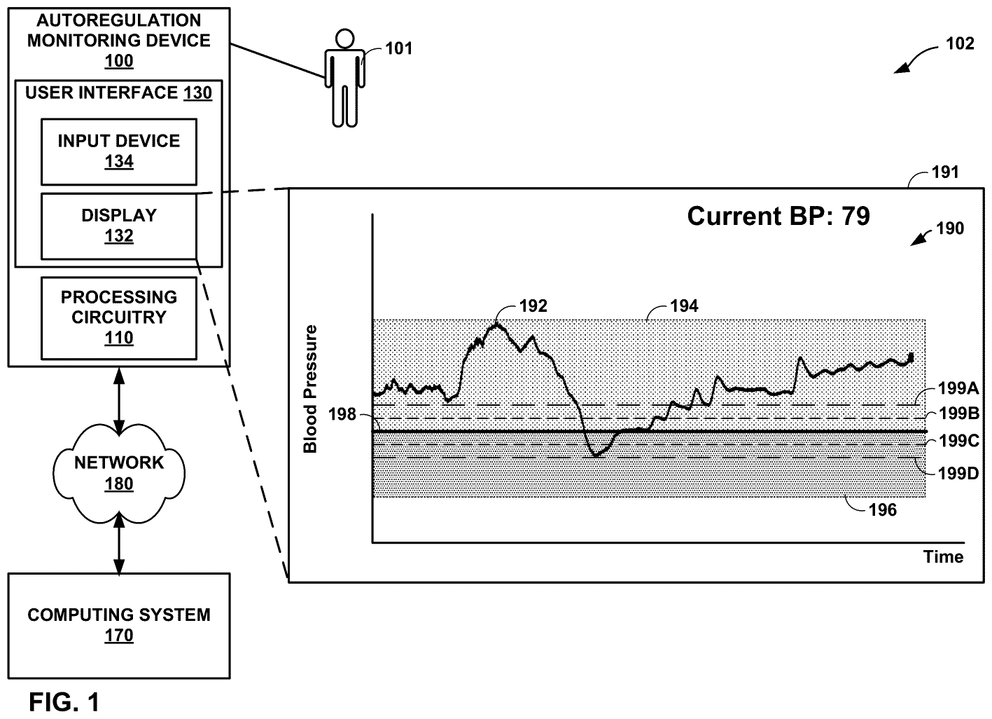

FIG. 1 illustrates an example environment 102 in which an example autoregulation monitoring device 100 may monitor the autoregulation status of a patient 101. As shown in FIG. 1, autoregulation monitoring device 100 may communicate with a computing system 170 via network 180.

Computing system 170 may be any suitable remote computing system, such as one or more desktop computers, laptop computers, mainframes, servers, cloud computing systems, virtual machines, etc. capable of sending and receiving information via network 180 to and from autoregulation monitoring devices, such as autoregulation monitoring device 100. In some examples, computing system 170 may represent a cloud computing system that provides one or more services via network 180. That is, in some examples, computing system 170 may be a distributed computing system in which one or more computing devices coordinate execution of threads to collaboratively perform one or more operations attributed to computing system 170. One or more computing devices, such as autoregulation monitoring device 100, may access the services provided by the cloud by communicating with computing system 170.

In some examples, computing system 170 may manage a fleet of autoregulation monitoring devices that include autoregulation monitoring device 100. Such a fleet of autoregulation monitoring devices may be located at an institution, such as a clinic or hospital, or may be distributed across different institutions, such as different hospitals and clinics. Computing system 170 may communicate with the fleet of autoregulation monitoring devices via network 180 to provide software updates, updated settings, and the like, and to monitor the operations of the fleet of autoregulation monitoring devices.

Network 180 may include a wide-area network such as the Internet, a local-area network (LAN), a personal area network (PAN) (e.g., Bluetooth®), an enterprise network, a wireless network, a cellular network, a telephony network, a Metropolitan area network (e.g., WIFI, WAN, WiMAX, etc.), one or more other types of networks, or a combination of two or more different types of networks (e.g., a combination of a cellular network and the Internet).

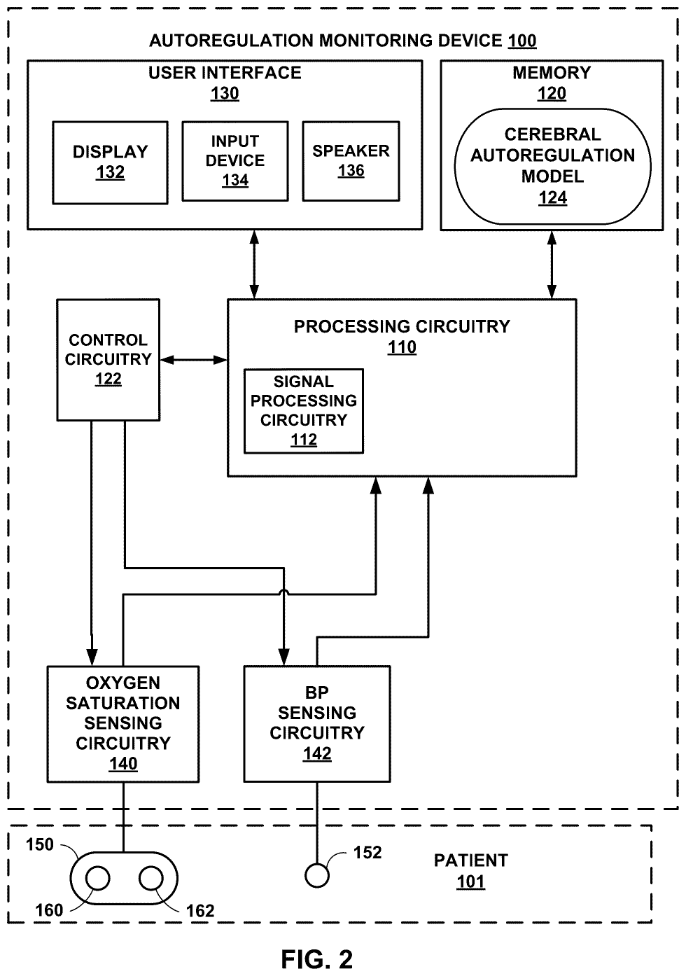

Autoregulation monitoring device 100 includes processing circuitry 110 and user interface 130. FIG. 1 illustrates just some of the components of autoregulation monitoring device 100, and the components of autoregulation monitoring device 100 are described in further detail below with respect to FIG. 2.

Processing circuitry 110, as well as other processors, processing circuitry, controllers, control circuitry, and the like, described herein, may include one or more processors. Processing circuitry 110 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 110 may include multiple components, such as any combination of one or more microprocessors, one or more DSPs, one or more ASICs, or one or more FPGAs, as well as other discrete or integrated logic circuitry, and/or analog circuitry.

User interface 130 may include a display 132, an input device 134, and a speaker 136. In some examples, user interface 130 may include fewer or additional components. User interface 130 may be configured to present information to a user (e.g., a clinician). For example, user interface 130 and/or display 132 may include a monitor, cathode ray tube display, a flat panel display such as a liquid crystal (LCD) display, a plasma display, a light emitting diode (LED) display, and/or any other suitable display. In some examples, display 132 and input device 134 may be integrated into a touchscreen that accepts touch input. In some examples, user interface 130 may be part of a multiparameter monitor (MPM) or other physiological signal monitor used in a clinical or other setting, a personal digital assistant, mobile phone, tablet computer, laptop computer, any other suitable computing device, or any combination thereof, with a built-in display or a separate display.

Processing circuitry 110 may be configured to output a graphical user interface (GUI) for display at display 132. That is, processing circuitry 110 may send instructions to display 132 that causes display 132 to display a GUI. The GUI may include information associated with the autoregulation status of patient 101, such as information associated with a cerebral autoregulation status of patient 101 and/or information associated with a non-cerebral autoregulation status of patient 101.

In the example of FIG. 1, processing circuitry 110 may output GUI 191 for display at display 132. GUI 191 may present information associated with the autoregulation status of patient 101 by including graph 190 of a blood pressure value 192 of patient 101 over time, as sensed by autoregulation monitoring device 100. Graph 190 includes an autoregulation threshold 198 that separates an intact autoregulation area 194 of the graph 190 and an impaired autoregulation area 196 of the graph 190.

Blood pressure value 192 of patient 101 may be the mean arterial pressure (MAP) of patient 101, which may be the average (i.e. mean) blood pressure in patient 101 during a single cardiac cycle. As such, in some examples, blood pressure value 192 may indicate a MAP for each cardiac cycle of patient 101 during a period of time.

Autoregulation threshold 198 may be presented in graph 190 as a line, such as a solid line as shown in FIG. 1, at a particular blood pressure value in graph 190. Autoregulation threshold 198 may be the LLA of patient 101, such that blood pressure values below autoregulation threshold 198 is below the LLA of patient 101 and the blood pressure values above autoregulation threshold 198 is above the LLA of patient 101. In some examples, autoregulation threshold 198 may be different from the LLA of patient 101, such as being the LLA of patient 101 plus or minus a specific offset value (e.g., the LLA of patient 101 plus 5 mmHg).

Intact autoregulation area 194 may be the area of graph 190 above autoregulation threshold 198 and impaired autoregulation area 196 may be the area of graph 190 below autoregulation threshold 198. In some examples, intact autoregulation area 194 and impaired autoregulation area 196 may be presented in different colors in graph 190. For example, intact regulation area 194 may be presented in green while impaired autoregulation area 196 may be presented in red. In some examples, graph 190 may include text labels that label intact autoregulation area 194 as intact and that label impaired autoregulation area 196 as impaired. In some examples, different portions of blood pressure value 192 in intact autoregulation area 194 and impaired autoregulation area 196 may be presented in different colors in graph 190. For example, portions of blood pressure value 192 in intact autoregulation area 194 may be presented in green while portions of blood pressure value 192 in impaired autoregulation area 196 may be presented in red.

Autoregulation threshold 198, intact autoregulation area 194, and impaired autoregulation area 196 in graph 190 enables graph 190 to provide information regarding the autoregulation status of patient 101 over time by graphically indicating whether the autoregulation status of patient 101 is intact or impaired over time. For example, graph 190 may indicate that the autoregulation status of patient 101 is intact when the blood pressure value 192 of patient 101 is in the intact autoregulation area 194, and graph 190 may indicate that the autoregulation status of patient 101 is impaired when the blood pressure value 192 of patient 101 is in the impaired autoregulation area 196.

A clinician may therefore view GUI 191 displayed by display 132, such as while a medical procedure is being performed on patient 101, to monitor the autoregulation status of patient 101 by monitoring whether the blood pressure value 192 of patient 101 is above the autoregulation threshold 198 and in the intact autoregulation area 194 or whether the blood pressure value 192 of patient 101 is below the autoregulation threshold 198 and in the impaired autoregulation area 194. The clinician may monitor the autoregulation status of patient 101 in order to determine whether to adjust medication being administered to patient 101. For example, the clinician may adjust the amount of anesthesia being administered to patient 101 to keep the blood pressure value 192 of patient 101 above autoregulation threshold 198 and within the intact autoregulation area 194.

In some examples, if processing circuitry 110 determines that the autoregulation status and of patient 101 is impaired, such as when blood pressure value 192 of patient 101 drops below autoregulation threshold 198, then processing circuitry 110 may present a notification indicating the impairment. The notification may include a visual, audible, tactile, or somatosensory notification (e.g., an alarm signal) indicative of the cerebral autoregulation status and/or the non-cerebral autoregulation status of patient 101. For example, processing circuitry 110 may output a visual warning in GUI 191 or may output, via speaker 136, an audible warning (e.g., a beep), to indicate that the autoregulation status and of patient 101 is impaired.

In some examples, a clinician may prefer to keep the blood pressure value 192 at, above, and/or below a blood pressure threshold that is offset from autoregulation threshold 198. For example, the clinician may prefer to keep the blood pressure value 192 of patient 101 above a blood pressure threshold that is offset by 5 millimeters of mercury (mmHg) above autoregulation threshold 198, or may prefer to keep the blood pressure value 192 of patient 101 above a blood pressure threshold that is offset by 5 mmHg below autoregulation threshold 198.

In accordance with aspects of this disclosure, graph 190 also includes autoregulation threshold offsets 199A-199D (hereafter “autoregulation threshold offsets 199”). Autoregulation threshold offsets 199 are offset from the autoregulation threshold 198 and from each other by a specific blood pressure offset, such as 5 mmHg, 10 mmHg, and the like. For example, given a specific blood pressure offset of 5 mmHg, autoregulation threshold offset 199B may be 5 mmHg above autoregulation threshold 198, and autoregulation threshold offset 199A may be 5 mmHg above autoregulation threshold offset 199B and 10 mmHg above autoregulation threshold 198. Similarly, autoregulation threshold offset 199C may be 5 mmHg below autoregulation threshold 198, and autoregulation threshold offset 199D may be 5 mmHg below autoregulation threshold offset 199C and 10 mmHg below autoregulation threshold 198.

In the example of FIG. 1, autoregulation threshold offsets 199 may be indicated in graph 190 as lines at associated blood pressure offsets from autoregulation threshold 198 and from each other. A clinician may therefore be able to view GUI 191 displayed at display 132 to view the blood pressure value 192 of patient 101 in relation to one or more autoregulation threshold offsets 199 indicated in graph 190. A clinician that prefers to keep the blood pressure value 192 of patient 101 above a particular autoregulation threshold offset 199, such as above autoregulation threshold offset 199B, may view GUI 191 to determine whether the blood pressure value 192 is close to dropping below autoregulation threshold offset 199B and may, in response to determining that the blood pressure value 192 is close to dropping below autoregulation threshold offset 199B, adjust the medication administered to patient 101 in order to increase the blood pressure value 192 to prevent the blood pressure value 192 from dropping below autoregulation threshold offset 199B.

In some examples, if processing circuitry 110 determines that the autoregulation status and of patient 101 is close to being impaired, then processing circuitry 110 may present a notification indicating that the autoregulation status of patient 101 is close to being impaired. For example, if processing circuitry 110 determines that blood pressure value 192 of patient 101 has dropped below the autoregulation threshold offset above autoregulation threshold 198 that is closest to autoregulation threshold 198, such as if processing circuitry 110 determines that blood pressure value 192 of patient 101 has dropped below autoregulation threshold offset 199B, processing circuitry 110 may present a notification indicating that the autoregulation status of patient 101 is close to being impaired.

The notification may include a visual, audible, tactile, or somatosensory notification (e.g., an alarm signal) indicating that the autoregulation status of patient 101 is close to being impaired. For example, processing circuitry 110 may output a visual warning in GUI 191 or may output, via speaker 136, an audible warning (e.g., a beep), to indicate that the autoregulation status of patient 101 is close to being impaired.

Graph 190 may indicate autoregulation threshold offsets 199 in various ways. As shown in FIG. 1, autoregulation threshold offsets 199 are presented as dotted lines, where different autoregulation threshold offsets 199 are presented as different patterns of dotted lines depending on the distance between each of the autoregulation threshold offsets 199 and autoregulation threshold 198. For example, autoregulation threshold offsets 199B and 199C are the same distance from autoregulation threshold 198 and may be represented using the same pattern of dotted lines, while autoregulation threshold offsets 199A and 199D are the same distance from autoregulation threshold 198 and may be represented using the same pattern of dotted lines which are different from the pattern of dotted lines representing autoregulation threshold offsets 199B and 199C. In other examples, different autoregulation threshold offsets 199 may be presented in graph 190 as different color gradients, different color bands, different color brightness, lines of different thicknesses, lines of different colors, and the like, to visually differentiate different autoregulation threshold offsets 199 from each other.

Processing circuitry 110 may be configured to determine the values of autoregulation threshold 198 and/or autoregulation threshold offsets 199 in graph 190 based on a variety of factors. For example, if autoregulation monitoring device 100 is part of an institution, such as a hospital and a clinic, processing circuitry 110 may determine institution-wide settings for the values of autoregulation threshold 198 and/or autoregulation threshold offsets 199, such as by reading such settings stored in memory of autoregulation monitoring device 100 or by receiving indications of such settings from computing system 170.

In some examples, processing circuitry 110 may determine the values of autoregulation threshold 198 and/or autoregulation threshold offsets 199 based on factors such as the identity of patient 101, the patient demographics of patient 101, medications that patient 101 is taking, the type of medical procedure being performed, whether the cerebral autoregulation status or a non-cerebral autoregulation status is being presented in GUI 191, and the like. In some examples, autoregulation monitoring device 100 may receive indications of the values of autoregulation threshold 198 and/or autoregulation threshold offsets 199 from computing system 170. That is, computing system 170 may determine the values of autoregulation threshold 198 and/or autoregulation threshold offsets 199 and may send indications of the values of autoregulation threshold 198 and/or autoregulation threshold offsets 199 to autoregulation monitoring device 100.

In some examples, a clinician may interact with user interface 130 to modify or otherwise adjust autoregulation threshold 198 and/or autoregulation threshold offsets 199. In examples where display 132 and input device 134 comprise a touchscreen, the clinician may interact with graph 190 displayed by display 132 by providing touch input, such as by performing tap gestures, drag gestures, and the like to adjust autoregulation threshold 198 and/or autoregulation threshold offsets 199. In some examples, GUI 191 may include one or more user interface elements, such as text fields, dropdown boxes, and the like with which a clinician may interact to adjust autoregulation threshold 198 and/or autoregulation threshold offsets 199.

By presenting graph 190 of blood pressure value 192 of patient 101 over time and by presenting an autoregulation threshold 198 as well as autoregulation threshold offsets 199, the techniques of this disclosure may provide more detailed information regarding the autoregulation status and blood pressure values of patient 101 to a clinician. For example, by presenting autoregulation threshold offsets 199 that are offset from each other and from autoregulation threshold 198 by a specific blood pressure offset, the techniques of this disclosure may enable a clinician to monitor the blood pressure value 192 of patient 101 against blood pressure levels other than autoregulation threshold 198, which may enable autoregulation monitoring device 100 to more accurately present the autoregulation status of patient 101 even in situations where autoregulation monitoring device 100 is unable to accurately determine the LLA of patient 101, which may lead to an improved medical outcome for patient 101.

Further, because autoregulation threshold 198 and autoregulation threshold offsets 199 may be adjusted via user input by a clinician, aspects of this disclosure may enable a clinician to personalize autoregulation threshold 198 and/or autoregulation threshold offsets 199 for a variety of different use cases, such as by personalizing autoregulation threshold 198 and/or autoregulation threshold offsets 199 for different patients, different types of medical procedures, different types of autoregulation statuses, and the like. Being able to personalize autoregulation threshold 198 and/or autoregulation threshold offsets 199 for different use cases may enable a clinician to tune autoregulation monitoring device 100 to more accurately present the autoregulation status of patient 101 even in situations where autoregulation monitoring device 100 is unable to accurately determine the LLA of patient 101, which may lead to an improved medical outcome for patient 101.

FIG. 2 is a conceptual block diagram illustrating an example autoregulation monitoring device 100. Autoregulation monitoring device 100 includes processing circuitry 110, memory 120, control circuitry 122, user interface 130, sensing circuitry 140 and 142, and sensing devices 150 and 152. In the example illustrated in FIG. 2, user interface 130 includes display 132, input device 134, and speaker 136, which may be any suitable audio device configured to generate and output a noise and include any suitable circuitry. In some examples, autoregulation monitoring device 100 may be configured to determine and display the cerebral autoregulation status of a patient 101, e.g., during a medical procedure or for more long-term monitoring, such as fetal monitoring. A clinician may receive information regarding the cerebral autoregulation status and/or a non-cerebral autoregulation status of a patient via user interface 130 and adjust treatment or therapy to patient 101 based on the autoregulation status information.

Control circuitry 122 may be operatively coupled processing circuitry 110. Control circuitry 122 is configured to control an operation of sensing devices 150 and 152. In some examples, control circuitry 122 may be configured to provide timing control signals to coordinate operation of sensing devices 150 and 152. For example, sensing circuitry 140 and 142 may receive from control circuitry 122 one or more timing control signals, which may be used by sensing circuitry 140 and 142 to turn on and off respective sensing devices 150 and 152. In some examples, processing circuitry 110 may use the timing control signals to operate synchronously with sensing circuitry 140 and 142. For example, processing circuitry 110 may synchronize the operation of an analog-to-digital converter and a demultiplexer with sensing circuitry 140 and 142 based on the timing control signals.

Memory 120 may be configured to store data, such as, for example, monitored physiological parameter values (including blood pressure values and/or oxygen saturation values), one or more cerebral autoregulation status values, one or more non-cerebral autoregulation status values, and/or one or more adjustment values, COx values, BVS values, HVx values, or any combination thereof. In some examples, data may be stored in memory 120 as one or more look-up tables or equations defining one or more associations (e.g., relationships) between stored data, such as, for example, associations between cerebral autoregulation status values and non-cerebral autoregulation status values. In some examples, memory 120 may store program instructions, such as neural network algorithms and/or finite element algorithms. The program instructions may include one or more program modules that are executable by processing circuitry 110. When executed by processing circuitry 110, such program instructions may cause processing circuitry 110 to provide the functionality ascribed to it herein. The program instructions may be embodied in software, firmware, and/or RAMware. Memory 120 may include any one or more of volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

User interface 130 may include a display 132, an input device 134, and a speaker 136, as described above with respect to FIG. 1. In some examples, processing circuitry 110 may be configured to present, by user interface 130, such as display 132, a graphical user interface to a user, such as graph 190 illustrated in FIG. 1. User interface 130 may also include means for projecting audio to a user, such as speaker 136.

In some examples, processing circuitry 110 may also receive input signals from additional sources (not shown), such as a user. For example, processing circuitry 110 may receive from input device 134, such as a keyboard, a mouse, a touch screen, buttons, switches, a microphone, a joystick, a touch pad, or any other suitable input device or combination of input devices, an input signal. The input signal may contain information about patient 101, such as physiological parameters, treatments provided to patient 101, or the like. Additional input signals may be used by processing circuitry 110 in any of the determinations or operations it performs in accordance with processing circuitry 110.

In some examples, processing circuitry 110 and user interface 130 may be part of the same device or supported within one housing (e.g., a computer or monitor). In other examples, processing circuitry 110 and user interface 130 may be separate devices configured to communicate through a wired connection or a wireless connection (e.g., a communication interface).

Oxygen saturation sensing circuitry 140 and blood pressure sensing circuitry (collectively, sensing circuitry 140 and 142) may be configured to receive physiological signals sensed by respective sensing devices 150 and 152 and communicate the physiological signals to processing circuitry 110. Sensing devices 150 and 152 may include any sensing hardware configured to sense a physiological parameter of a patient, such as, but not limited to, one or more electrodes, optical receivers, blood pressure cuffs, or the like. The sensed physiological signals may include signals indicative of physiological parameters from a patient, such as, but not limited to, blood pressure, regional oxygen saturation, blood volume, heart rate, and respiration. For example, sensing circuitry 140 and 142 may include, but are not limited to, blood pressure sensing circuitry, oxygen saturation sensing circuitry, blood volume sensing circuitry, heart rate sensing circuitry, temperature sensing circuitry, electrocardiography (ECG) sensing circuitry, electroencephalogram (EEG) sensing circuitry, or any combination thereof.

In some examples, sensing circuitry 140 and 142 and/or processing circuitry 110 may include signal processing circuitry 112 configured to perform any suitable analog conditioning of the sensed physiological signals. For example, sensing circuitry 140 and 142 may communicate to processing circuitry 110 an unaltered (e.g., raw) signal. Processing circuitry 110, e.g., signal processing circuitry 112, may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals may be processed by an analog-to-digital converter of signal processing circuitry 112 to convert the conditioned analog signals into digital signals. In some examples, signal processing circuitry 112 may operate on the analog or digital form of the signals to separate out different components of the signals. In some examples, signal processing circuitry 112 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. In some examples, signal processing circuitry 112 may decrease the number of samples in the digital detector signals. In some examples, signal processing circuitry 112 may remove dark or ambient contributions to the received signal. Additionally or alternatively, sensing circuitry 140 and 142 may include signal processing circuitry 112 to modify one or more raw signals and communicate to processing circuitry 110 one or more modified signals.

Oxygen saturation sensing device 150 is configured to generate an oxygen saturation signal indicative of blood oxygen saturation within the venous, arterial, and/or capillary systems within a region of patient 101. For example, oxygen saturation sensing device 150 may be configured to be placed on the skin of patient 101 to determine regional oxygen saturation of a particular tissue region, e.g., the frontal cortex or another cerebral location of patient 101. Oxygen saturation sensing device 150 may include emitter 160 and detector 162. Emitter 160 may include at least two light emitting diodes (LEDs), each configured to emit at different wavelengths of light, e.g., red or near infrared light. As used herein, the term “light” may refer to energy produced by radiative sources and may include any wavelength within one or more of the ultrasound, radio, microwave, millimeter wave, infrared, visible, ultraviolet, gamma ray or X-ray electromagnetic radiation spectra. In some examples, light drive circuitry (e.g., within sensing device 150, sensing circuitry 140, control circuitry 122, and/or processing circuitry 110) may provide a light drive signal to drive emitter 160 and to cause emitter 160 to emit light. In some examples, the LEDs of emitter 160 emit light in the range of about 600 nanometers (nm) to about 1000 nm. In a particular example, one LED of emitter 160 is configured to emit light at about 730 nm and the other LED of emitter 160 is configured to emit light at about 810 nm. Other wavelengths of light may be used in other examples.

Detector 162 may include a first detection element positioned relatively “close” (e.g., proximal) to emitter 160 and a second detection element positioned relatively “far” (e.g., distal) from emitter 160. In some examples, the first detection elements and the second detection elements may be chosen to be specifically sensitive to the chosen targeted energy spectrum of light source 160. Light intensity of multiple wavelengths may be received at both the “close” and the “far” detector 162. For example, if two wavelengths are used, the two wavelengths may be contrasted at each location and the resulting signals may be contrasted to arrive at an oxygen saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium). In operation, light may enter detector 162 after passing through the tissue of patient 101, including skin, bone, other shallow tissue (e.g., non-cerebral tissue and shallow cerebral tissue), and/or deep tissue (e.g., deep cerebral tissue). Detector 162 may convert the intensity of the received light into an electrical signal. The light intensity may be directly related to the absorbance and/or reflectance of light in the tissue. Surface data from the skin and skull may be subtracted out, to generate an oxygen saturation signal for the target tissues over time.

Oxygen saturation sensing device 150 may provide the oxygen saturation signal to processing circuitry 110 or to any other suitable processing device to enable evaluation of an autoregulation status of patient 101. Additional example details of determining oxygen saturation based on light signals may be found in commonly assigned U.S. Pat. No. 9,861,317, which issued on Jan. 9, 2018, and is entitled “Methods and Systems for Determining Regional Blood Oxygen Saturation.”

In operation, blood pressure sensing device 152 and oxygen saturation sensing device 150 may each be placed on the same or different parts of the body of patient 101. For example, blood pressure sensing device 152 and oxygen saturation sensing device 150 may be physically separate from each other and may be separately placed on patient 101. As another example, blood pressure sensing device 152 and oxygen saturation sensing device 150 may in some cases be part of the same sensor or supported by a single sensor housing. For example, blood pressure sensing device 152 and oxygen saturation sensing device 150 may be part of an integrated oximetry system configured to non-invasively measure blood pressure (e.g., based on time delays in a plethysmography (PPG) signal) and regional oxygen saturation. One or both of blood pressure sensing device 152 or oxygen saturation sensing device 150 may be further configured to measure other parameters, such as hemoglobin, respiratory rate, respiratory effort, heart rate, saturation pattern detection, response to stimulus such as bispectral index (BIS) or electromyography (EMG) response to electrical stimulus, or the like. While an example autoregulation monitoring device 100 is illustrated in FIG. 2, the components illustrated in FIG. 2 are not intended to be limiting. Additional or alternative components and/or implementations may be used in other examples.

Blood pressure sensing device 152 may be any sensor or device configured to generate a blood pressure signal indicative of a blood pressure of patient 101 at acquisition site 103. For example, blood pressure sensing device 152 may include a blood pressure cuff configured to non-invasively monitoring blood pressure, a sensor configured to noninvasively generate a PPG signal, or an arterial line for invasively monitoring blood pressure in an artery of patient 101. In some examples, the blood pressure signal may include at least a portion of a waveform of the acquisition blood pressure. In some examples, acquisition site 103 may include at least one of a femoral artery of patient 101, a radial artery of patient 101, a dorsalis pedis artery of patient 101, a brachial artery of patient 101, or combinations thereof. In some examples, blood pressure sensing device 152 may include a plurality of blood pressure sensing devices. For example, each blood pressure sensing device of the plurality of blood pressure sensing devices may be configured to obtain a respective blood pressure of patient 101 at a respective acquisition site of a plurality of acquisition sites. The plurality of acquisition sites may include similar or different arteries of patient 101.

In some examples, blood pressure sensing device 152 may include one or more pulse oximetry sensors. The acquisition blood pressure may be derived by processing time delays between two or more characteristic points within a single PPG signal obtained from a single pulse oximetry sensor. Additional example details of deriving blood pressure based on a comparison of time delays between certain components of a single PPG signal obtained from a single pulse oximetry sensor are described in commonly assigned U.S. Patent Application Publication No. 2009/0326386 filed Sep. 30, 2008, entitled “Systems and Methods for Non-Invasive Blood Pressure Monitoring.” In other cases, the blood pressure of patient 101 may be continuously, non-invasively monitored via multiple pulse oximetry sensors placed at multiple locations on patient 101. As described in commonly assigned U.S. Pat. No. 6,599,251, issued Jul. 29, 2003, entitled “Continuous Non-invasive Blood Pressure Monitoring Method and Apparatus,” multiple PPG signals may be obtained from the multiple pulse oximetry sensors, and the PPG signals may be compared against one another to estimate the blood pressure of patient 101.

Regardless of its form, blood pressure sensing device 152 may be configured to generate a blood pressure signal indicative of a blood pressure of patient 101 (e.g., arterial blood pressure) over time. In examples in which blood pressure sensing device 152 includes a plurality of blood pressure sensing devices, the blood pressure signal may include a plurality of blood pressure signals, each indicative of a blood pressure of patient 101 at a respective acquisition site. Blood pressure sensing device 152 may provide the blood pressure signal to sensing circuitry 142, processing circuitry 110, or to any other suitable processing device to enable evaluation of the autoregulation status of patient 101.

Processing circuitry 110 may be configured to receive one or more signals generated by sensing devices 150 and 152 and sensing circuitry 140 and 142. The physiological signals may include a blood pressure signal indicative of a blood pressure of patient 101 and/or an oxygen saturation signal indicative of an oxygen saturation of patient 101. After receiving one or more signals, processing circuitry 110 may be configured to determine a metric (e.g., a numerical value or qualitative information) indicative of the autoregulation status of patient 101, such as a cerebral autoregulation status value. The cerebral autoregulation status value may be based on the blood pressure signal and the oxygen saturation signal. For example, processing circuitry 110 may determine a correlation index (e.g., COx, HVx) or other measure of autoregulation, such as based on co-trending of blood pressure and blood oxygen saturation, (e.g., based on a comparison of blood pressure gradients and oxygen saturation gradients), based on the blood pressure signal and the oxygen saturation signal. In other examples, processing circuitry 110 may determine the correlation index based on additional or alternative physiological parameters (e.g., physiological signals), such as, for example, a blood volume value or a gradients measure.

In some examples, processing circuitry 110 may determine an estimate of an LLA based on the lowest blood pressure value at which the expected value of COx is less than a threshold value, such as 0.5, 0.4, 0.3, 0.2, 0.1, or 0.0. Using this threshold value (e.g., 0.5, 0.4, 0.3, 0.2, 0.1, or 0.0), processing circuitry 110 can determine where there is a distinct change in a correlation between the blood pressure and the oxygen saturation, such as an oxygen saturation versus blood pressure curve. This distinct change may correspond to a distinct step down in the plot of COx or HVx versus blood pressure. Similarly, processing circuitry 110 may determine an estimate of a ULA based on the highest blood pressure value at which the expected value of COx is less than a threshold value. Additional example details of determining limits of autoregulation (Las) and cerebral autoregulation status may be found in commonly assigned U.S. Patent Application Publication No. 2018/0014791, filed on Jul. 13, 2017, entitled “Systems and Methods of Monitoring Autoregulation”; commonly assigned U.S. Patent Application Publication No. 2018/0049649 filed on Aug. 1, 2017, entitled “System and Method for Identifying Blood Pressure Zones During Autoregulation Monitoring”; commonly assigned U.S. Patent Application Publication No. 2016/0367197 filed on Dec. 22, 2016, entitled “Systems and Methods of Reducing Signal Noise When Monitoring Autoregulation”; and commonly assigned U.S. patent application Ser. No. 15/962,438 filed on Apr. 25, 2018, entitled “Determining Changes to Autoregulation.” In some examples, processing circuitry 110 may determine that a patient has intact autoregulation in response to determining that the blood pressure of patient 101 is greater than an LLA and less than an ULA (e.g., the blood pressure is between the limits of autoregulation).

In some examples, processing circuitry 110 may be configured to determine a non-cerebral autoregulation status value based on the cerebral autoregulation status value, such as by adding a specific offset value to a determined cerebral autoregulation status value to generate the non-cerebral autoregulation status value. Such a specific offset value may differ depending on the organ (e.g., kidneys, heart, etc.) for which the autoregulation status value is being determined. Example details of determining a non-cerebral autoregulation status value based on the cerebral autoregulation status value may be found in commonly assigned U.S. Pat. No. 10,932,673, issued on Mar. 2, 2021, entitled “Non-Cerebral Organ Autoregulation Status Determination.”

In some examples, processing circuitry 110 may be configured to display, at display 132, a graph of a blood pressure value of patient 101 over time. The graph may indicate the autoregulation status of patient 101 by including an autoregulation threshold that separates an intact autoregulation region from an impaired autoregulation region, thereby enabling a clinician to determine, based on whether the blood pressure value of patient 101 is in the intact autoregulation region or in the impaired autoregulation region, whether the autoregulation status of patient 101 is intact or impaired.

In some examples, the graph displayed at display 132 may also include autoregulation threshold offsets above and/or below the autoregulation threshold, thereby enabling a clinician to make a finer grained determination of the autoregulation status of patient 101. In examples where a clinician is attempting to keep the blood pressure of patient 101 above a specific blood pressure level that is different from the autoregulation threshold, the clinician may use an autoregulation threshold offset as a visual aid in order to keep the blood pressure of patient 101 above the specific blood pressure level.

In some examples, autoregulation monitoring device 100, e.g., processing circuitry 110 or user interface 130, may include a communication interface to enable autoregulation monitoring device 100 to exchange information with external devices (e.g., computing system 170). The communication interface may include any suitable hardware, software, or both, which may allow autoregulation monitoring device 100 to communicate with electronic circuitry, a device, a network (e.g., network 180), a server or other workstations, a display, or any combination thereof. For example, processing circuitry 110 may receive blood pressure values, oxygen saturation values, or predetermined data, such as predetermined autoregulation threshold offsets, predetermined autoregulation thresholds, and the like from an external device via the communication interface.

The components of autoregulation monitoring device 100 that are illustrated and described as separate components are illustrated and described as such for illustrative purposes only. In some examples the functionality of some of the components may be combined in a single component. For example, the functionality of processing circuitry 110 and control circuitry 122 may be combined in a single processor system. Additionally, in some examples the functionality of some of the components of autoregulation monitoring device 100 illustrated and described herein may be divided over multiple components. For example, some or all of the functionality of control circuitry 122 may be performed in processing circuitry 110, or sensing circuitry 140 and 142. In other examples, the functionality of one or more of the components may be performed in a different order or may not be required.

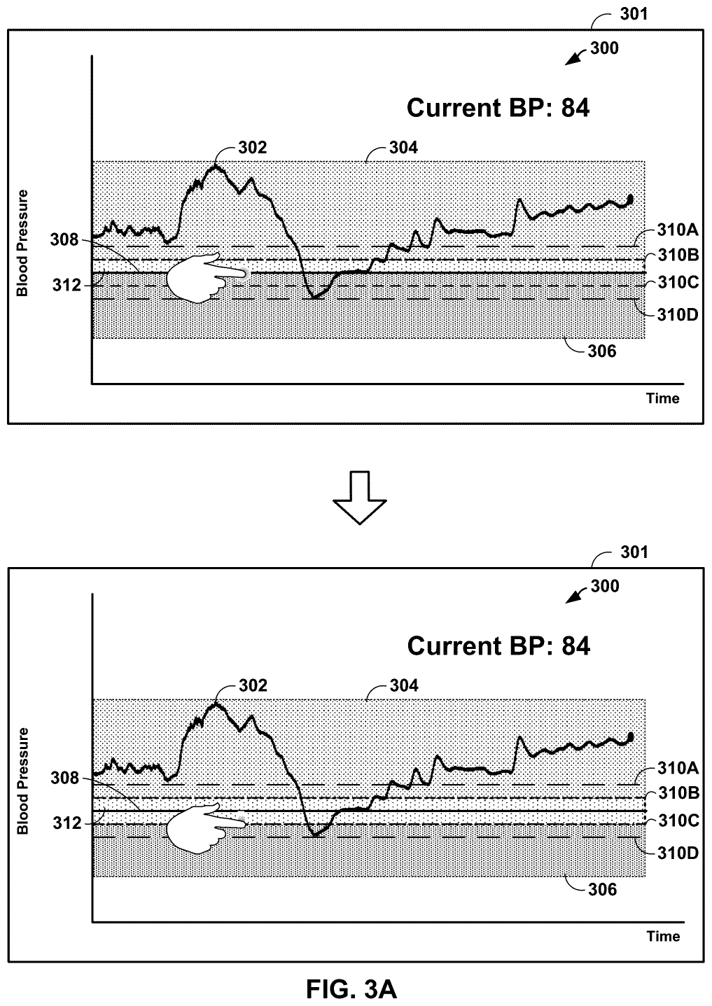

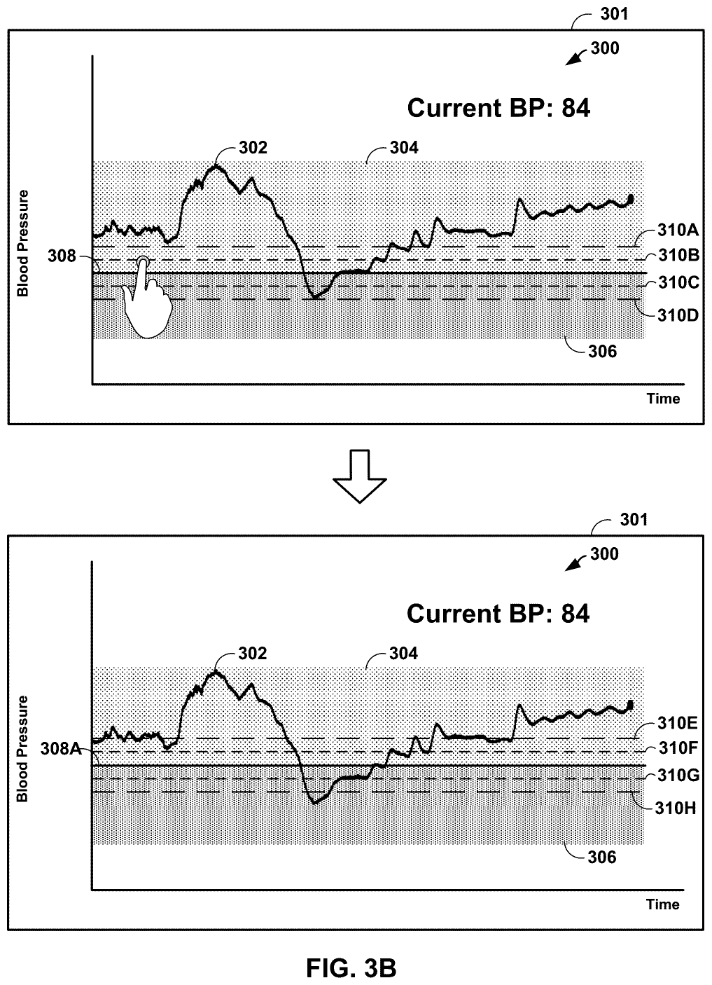

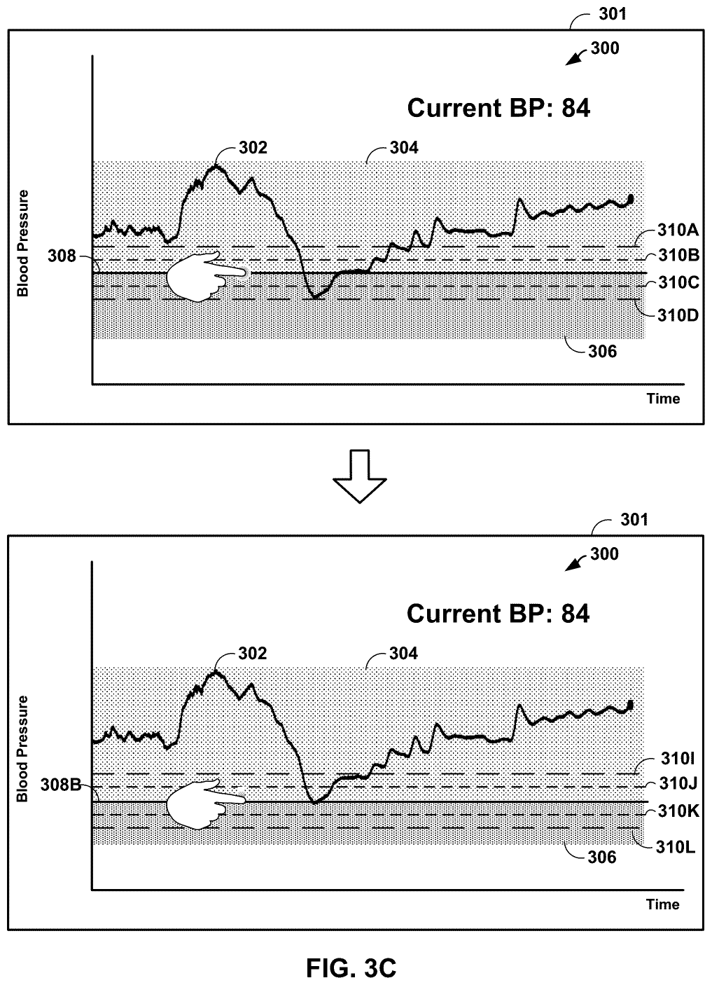

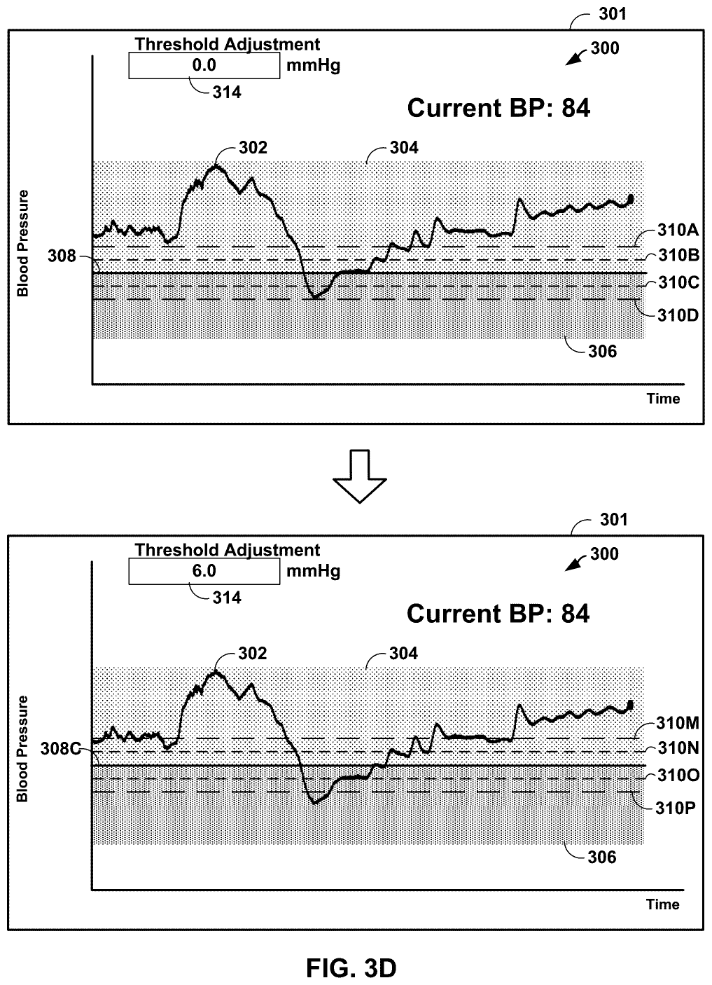

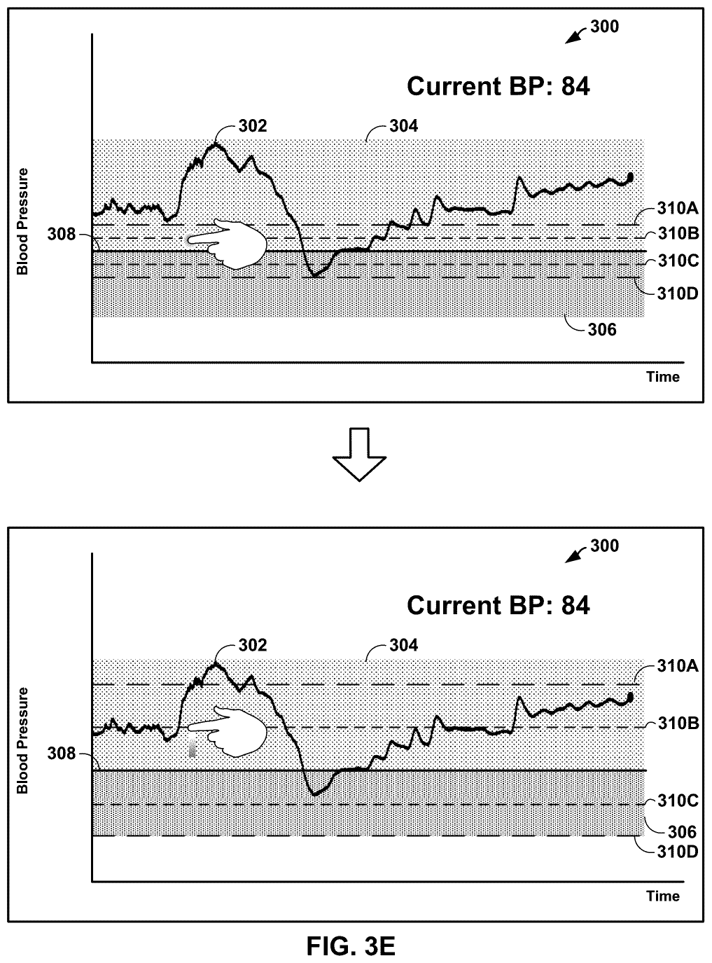

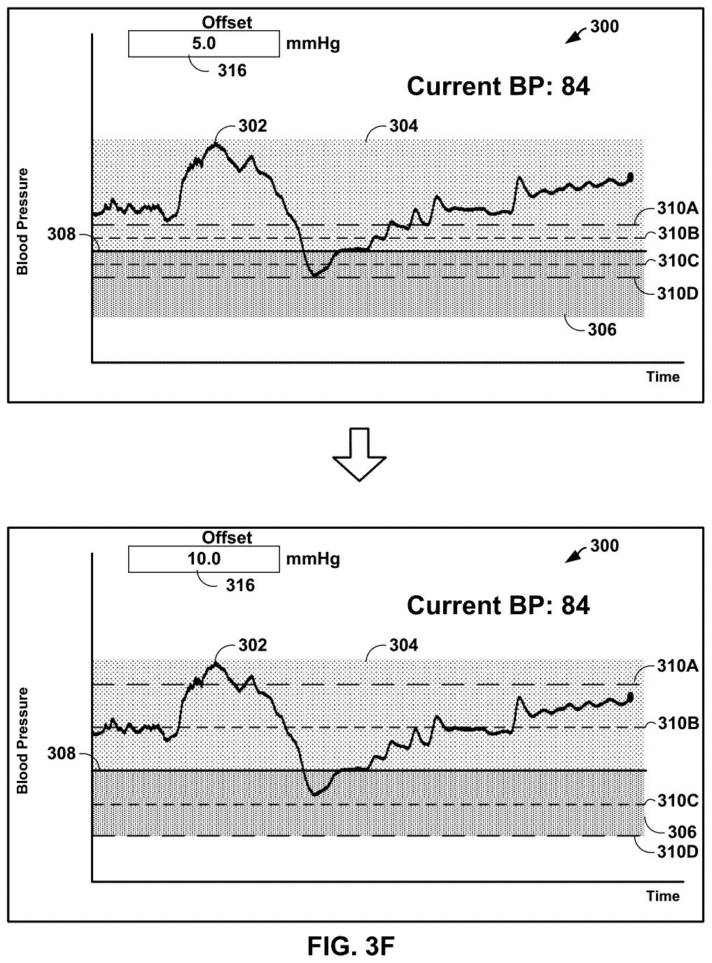

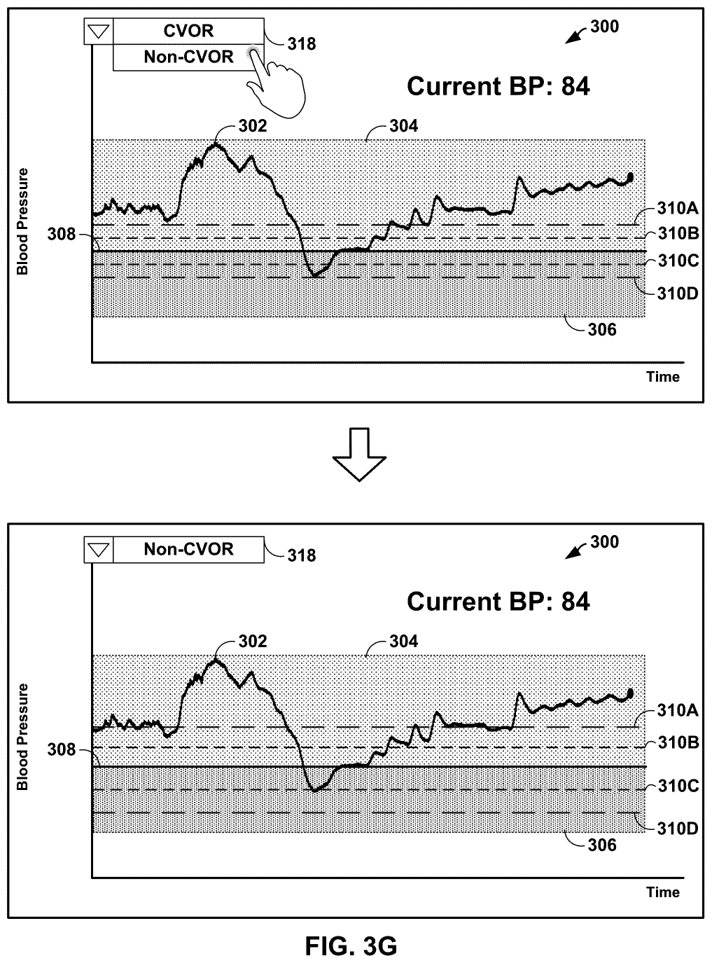

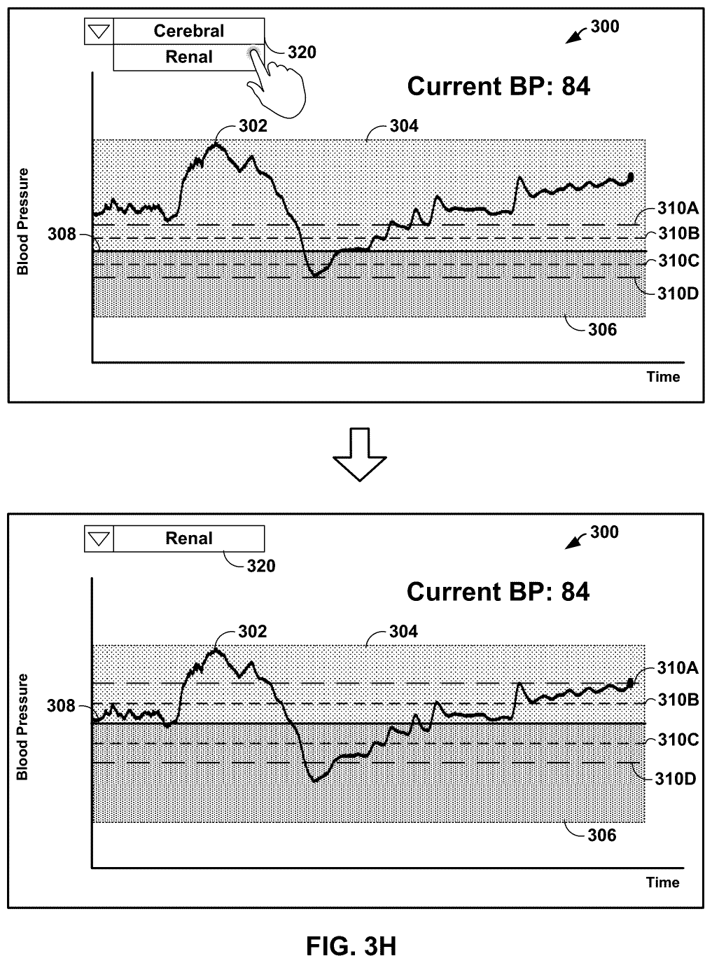

FIGS. 3A-3H illustrate example graphical user interfaces outputted by an example autoregulation monitoring device. FIGS. 3A-3H are described in the context of the autoregulation monitoring device 100 of FIGS. 1 and 2.

As shown in FIG. 3A, processing circuitry 110 may output, for display at display 132, GUI 301 that includes graph 300 of the blood pressure value 302 of patient 101 over time. Graph 300 includes autoregulation threshold 308 that separates intact autoregulation area 304 of graph 300 from impaired autoregulation area 306 of graph 300. Graph also includes autoregulation threshold offsets 310A-310D (hereafter “autoregulation threshold offsets 310”).

A clinician may prefer to keep blood pressure value 302 of patient 101 within a specific range of blood pressure values, such as between autoregulation threshold 308 and one of autoregulation threshold offsets 310 or between two of autoregulation threshold offsets 310. As such, autoregulation monitoring device 100 may output, in graph 300, autoregulation threshold offset area 312 that may be bounded by autoregulation threshold 308 and one of autoregulation threshold offsets 310 or by two of autoregulation threshold offsets 310.

Autoregulation monitoring device 100 may visually highlight autoregulation threshold offset area 312 in graph 300 in ways that distinguish autoregulation threshold offset area 312 from intact regulation area 304 and impaired autoregulation area 306, which may make it easier for clinicians to view GUI 301 to determine whether blood pressure value 302 is within autoregulation threshold offset area 312. For example, autoregulation threshold offset area 312 may be presented in a different color (e.g., yellow) in graph 300 than intact regulation area 304 and impaired autoregulation area 306. In another example, graph 300 may include a border (e.g., a solid line) around impaired autoregulation area 306 that enables clinicians to more easily be able to determine, at a glance of display 132, whether blood pressure value 302 is within autoregulation threshold offset area 312.

In the example of FIG. 3A, autoregulation threshold offset area 312 is bound by autoregulation threshold offset 310B and autoregulation threshold 308. Autoregulation threshold offset area 312 bound by autoregulation threshold offset 310B and autoregulation threshold 308 in graph 300 is visually distinct from intact regulation area 304 and impaired autoregulation area 306, such as by being presented in a different color than intact regulation area 304 and impaired autoregulation area 306.

Processing circuitry 110 is configured to determine whether to output autoregulation threshold 308 and to determine the bounds of autoregulation threshold 308 via any suitable technique. In some examples, a clinician may provide user input at input device 134 to select an option presented by autoregulation monitoring device 100 to turn on autoregulation threshold offset area 312 in graph 300, and processing circuitry 110 is configured to, in response to receiving, from input device 134, an indication of user input that corresponds to selection of the option to turn on autoregulation threshold offset area 312, output graph 300 that includes autoregulation threshold offset area 312. Similarly, a clinician may provide user input at input device 134 to select an option presented by autoregulation monitoring device 100 to specify the upper and/or lower bounds of autoregulation threshold offset area 312 in graph 300, and processing circuitry 110 is configured to, in response to receiving, from input device 134, one or more indications of user input that corresponds to specifying the upper and/or lower bounds of autoregulation threshold offset area 312, output graph 300 that includes autoregulation threshold offset area 312 having the specified upper and/or lower bounds.

In some examples, processing circuitry 110 is configured to receive, from computing system 170 an indication of whether to output autoregulation threshold offset area 312 and/or one or more indications of an upper bound and/or a lower bound of autoregulation threshold offset area 312. Processing circuitry 110 may, in response to receiving an indication to output autoregulation threshold offset area 312 from computing system 170 and/or one or more indications of an upper bound and/or a lower bound of autoregulation threshold offset area 312, output graph 300 that includes autoregulation threshold offset area 312 bounded by the indicated upper bound and/or indicated lower bound.

In some examples, processing circuitry 110 is configured to determine whether to output autoregulation threshold offset area 312 and/or to determine an upper bound and/or a lower bound of autoregulation threshold offset area 312 based on one or more factors, such as the identity of patient 101, demographics information (e.g., age, weight, comorbidities, etc.) of patient 101, the type of medical procedure being performed, whether the autoregulation status of patient 101 indicated in graph 300 is the cerebral autoregulation status of patient 101 or a non-cerebral autoregulation status of patient 101, the time of day, the particular medical institution at which autoregulation monitoring device 100 is situated, or any other factors. For example, different types of surgeries may be associated with different autoregulation threshold offset area having different upper and/or lower bounds, and processing circuitry 110 may be configured to determine to output autoregulation threshold offset area 312 and to determine the upper bound and/or lower bound of autoregulation threshold offset area 312 based on the type of surgery being performed on patient 101.

In some examples, a clinician may provide user input to adjust the bounds of autoregulation threshold offset area 312 in graph 300 that is displayed by display 132. Processing circuitry 110 may, in response to receiving, from user interface 130, an indication of user input that corresponds to adjusting an upper bound and/or a lower bound of autoregulation threshold offset area 312, adjust the upper bound and/or lower bound of display 132 according to the user input.

In the example where display 132 is a touchscreen, a clinician may provide touch input at display 132, such as in the form of a drag gesture, to adjust the upper and/or lower bounds of autoregulation threshold offset area 312 by dragging the upper and/or lower bounds of autoregulation threshold offset area 312. For example, if autoregulation threshold offset area 312 has an upper bound of autoregulation threshold offset 310B and a lower bound of autoregulation threshold 308, the clinician may adjust the lower bound of autoregulation threshold 308 by performing a drag gesture at display 132 to drag the lower bound of autoregulation threshold offset area 312 to an updated lower bound position, such as by dragging the lower bound of autoregulation threshold offset area 312 from autoregulation threshold 308 to autoregulation threshold offset 310C. Processing circuitry 110 may be configured to, in response to receiving, from user interface 130 an indication of a drag gesture perform by the clinician to drag lower bound of autoregulation threshold offset area 312 from autoregulation threshold 308 to autoregulation threshold offset 310C, update autoregulation threshold offset area 312 in graph 300 to have an upper bound of autoregulation threshold offset 310B and a lower bound of autoregulation threshold offset 310C, as shown in FIG. 3A.

Processing circuitry 110 may be configured to output a notification when blood pressure value 302 of patient 101 is outside of autoregulation threshold offset area 312. Such a notification may include a visual, audible, tactile, or somatosensory notification (e.g., an alarm signal) indicative of blood pressure value 302 being outside of autoregulation threshold offset area 312. For example, processing circuitry 110 may output a visual warning in GUI 191 or may output, via speaker 136, an audible warning (e.g., a beep), to indicate that blood pressure value 302 of patient 101 is outside of autoregulation threshold offset area 312.

In some examples, a user of autoregulation monitoring device 100, such as a clinician, may interact with a GUI displayed at display 132 to adjust how the autoregulation status of patient 101 is presented in the GUI. As shown in FIG. 3B, processing circuitry 110 may output, for display at display 132, GUI 301 that includes graph 300 of the blood pressure value 302 of patient 101 over time. Graph 300 includes autoregulation threshold 308 that separates intact autoregulation area 304 of graph 300 from impaired autoregulation area 306 of graph 300. Graph also includes autoregulation threshold offsets 310A-310D (hereafter “autoregulation threshold offsets 310”).

A clinician may provide user input at user interface 130 (e.g., at display 132 and/or input device 134) to interact with elements of graph 300 in GUI 301, such as by selecting, tapping, dragging, or otherwise directly interacting with elements of graph 300 to adjust autoregulation threshold 308, autoregulation threshold offsets 310, or other elements presented in graph 300. That is, the clinician may adjust the blood pressure value associated with autoregulation threshold 308 and/or the blood pressure values associated with autoregulation threshold offsets 310. For example, a clinician may, in examples where display 132 is a touchscreen, provide touch input at display 132 to interact with elements of graph 300. In another example, a clinician may use input device 134, which may be a mouse or a trackpad, to interact with elements of graph 300.

A clinician may provide user input at user interface 130 to select an autoregulation threshold offset out of autoregulation threshold offsets 310 to update autoregulation threshold 308 to a blood pressure value that corresponds to the selected autoregulation threshold offset. For example, processing circuitry 110 may be configured to receive, from user interface 130, an indication of the user input that corresponds to selection of an autoregulation threshold offset and may be configured to, in response, update autoregulation threshold 308 to a blood pressure value that corresponds to the selected autoregulation threshold offset, such as by updating autoregulation threshold 308 to be the sum of the blood pressure value (e.g., in mmHg) associated with the selected autoregulation threshold offset and the value of autoregulation threshold 308.

In the example of FIG. 3B, a clinician may provide user input to select autoregulation threshold offset 310B, such as by performing a tap gesture at a location of display 132 that corresponds to autoregulation threshold offset 310B to select autoregulation threshold offset 310B. Processing circuitry 110 may be configured to receive, from user interface 130, an indication of the user input that corresponds to selection of autoregulation threshold offset 310B and may be configured to, in response, update autoregulation threshold 308 to be the sum of the blood pressure value associated with autoregulation threshold offset 310B and the value of autoregulation threshold 308 to generate updated autoregulation threshold 308A.

Processing circuitry 110 may therefore be configured to update graph 300 to include updated autoregulation threshold 308A that is associated with a different blood pressure value than autoregulation threshold 308. Processing circuitry 110 may also be configured to update intact regulation area 304 and impaired autoregulation area 306 separated by updated autoregulation threshold 308A, such that intact regulation area 304 is the area above the updated autoregulation threshold 308A in graph 300 and impaired autoregulation area 306 is the area below the updated autoregulation threshold 308A in graph 300. For example, if the updated autoregulation threshold 308A is associated with a higher blood pressure value in graph 300 compared to autoregulation threshold 308, processing circuitry 110 may be configured to shrink intact regulation area 304 so that no portion of intact regulation area 304 is below the updated autoregulation threshold 308A, and processing circuitry 110 may be configured to expand impaired autoregulation area 306 to extend impaired autoregulation area 306 to the updated autoregulation threshold 308A in graph 300.

Processing circuitry 110 may be configured to, in response to updating autoregulation threshold 308 in graph 300, also adjust autoregulation threshold offsets 310 based on the blood pressure value of updated autoregulation threshold 308A in 300. In the example of FIG. 3B, processing circuitry 110 may be configured to update graph 300 to include autoregulation threshold offsets 310E-310H that are offset from each other and from the updated autoregulation threshold 308A by a specified blood pressure offset.

In the example of FIG. 3C, a clinician may provide user input in the form of a drag gesture, such as at display 132, to select and drag autoregulation threshold 308 to a location in graph 300 associated with a particular blood pressure value in order to update the blood pressure value associated with autoregulation threshold 308 to a blood pressure value that corresponds to the particular blood pressure value. For example, processing circuitry 110 may be configured to receive, from user interface 130, an indication of the user input that corresponds to the drag gesture that drags autoregulation threshold 308 to a location in graph 300 associated with a particular blood pressure value, and processing circuitry 110 may be configured to, in response, update autoregulation threshold 308 to a blood pressure value that corresponds to the particular blood pressure value, such as by setting autoregulation threshold 308 to be the particular blood pressure value, such as by generating an updated autoregulation threshold 308B that is set to the particular blood pressure value.

Processing circuitry 110 may therefore be configured to update graph 300 to include updated autoregulation threshold 308B that is associated with a different blood pressure value than autoregulation threshold 308. Processing circuitry 110 may also be configured to update intact regulation area 304 and impaired autoregulation area 306 separated by updated autoregulation threshold 308B, such that intact regulation area 304 is the area above the updated autoregulation threshold 308B in graph 300 and impaired autoregulation area 306 is the area below the updated autoregulation threshold 308B in graph 300. For example, if the updated autoregulation threshold 308B is associated with a lower blood pressure value in graph 300 compared to autoregulation threshold 308, processing circuitry 110 may be configured to shrink impaired regulation area 306 so that no portion of impaired regulation area 306 is above the updated autoregulation threshold 308B, and processing circuitry 110 may be configured to expand intact autoregulation area 304 to extend intact regulation area 304 to the updated autoregulation threshold 308B in graph 300.

Processing circuitry 110 may be configured to, in response to updating autoregulation threshold 308 to updated autoregulation threshold 308B in graph 300, also adjust autoregulation threshold offsets 310 based on the blood pressure value of updated autoregulation threshold 308B in 300. In the example of FIG. 3C, processing circuitry 110 may be configured to update graph 300 to include autoregulation threshold offsets 310I-310L that are offset from each other and from the updated autoregulation threshold 308B by a specified blood pressure offset.

In the example of FIG. 3D, GUI 301 includes a user interface element, such as text field 314, that enables a clinician to input a threshold adjustment value to adjust autoregulation threshold 308. For example, the clinician may input a positive threshold adjustment value to increase autoregulation threshold 308 or may input a negative threshold adjustment value to decrease autoregulation threshold 308.

For example, processing circuitry 110 may be configured to receive, from user interface 130, an indication of the user input that corresponds to input of a threshold adjustment value in text field 314 and may be configured to, in response, update autoregulation threshold 308 to a blood pressure value that corresponds to the threshold adjustment value, such as by adding the threshold adjustment value inputted by the clinician to autoregulation threshold 308 to generate updated autoregulation threshold 308C. In the example of FIG. 3D, the clinician may input a threshold adjustment value of 6.0 into text field 314 and processing circuitry 110 may be configured to, in response, add the threshold adjustment value of 6.0 to autoregulation threshold 308 to generate updated autoregulation threshold 308C.

Processing circuitry 110 may therefore be configured to update graph 300 to include updated autoregulation threshold 308C that is associated with a different blood pressure value than autoregulation threshold 308. Processing circuitry 110 may also be configured to update intact regulation area 304 and impaired autoregulation area 306 separated by updated autoregulation threshold 308C, such that intact regulation area 304 is the area above the updated autoregulation threshold 308C in graph 300 and impaired autoregulation area 306 is the area below the updated autoregulation threshold 308C in graph 300.

Processing circuitry 110 may be configured to, in response to updating autoregulation threshold 308 in graph 300 to updated autoregulation threshold 308C, also adjust autoregulation threshold offsets 310 based on the blood pressure value of updated autoregulation threshold 308B in 300. In the example of FIG. 3C, processing circuitry 110 may be configured to update graph 300 to include autoregulation threshold offsets 310M-310P that are offset from each other and from the updated autoregulation threshold 308C by a specified blood pressure offset.

In some examples, processing circuitry 110 may be configured to limit the amount of adjustment that can be made via user input (e.g., via the drag gesture) to autoregulation threshold 308. For example, processing circuitry 110 may be configured to limit the amount of adjustment that can be made to no more than 5 mmHg, no more than 10 mmHg, and the like. In another example, processing circuitry 110 may be configured to prevent the clinician from adjusting autoregulation threshold 308 to being below the LLA of patient 101. Thus, processing circuitry processing circuitry 110 may be configured to prevent the clinician from providing user input to increase or decrease autoregulation threshold 308 by more than a specific adjustment value (e.g., 5 mmHg, 10 mmHg, etc.) and/or to prevent the clinician from adjusting autoregulation threshold 308 to being below the LLA of patient 101.

In some examples, a clinician may provide user input at user interface 130 (e.g., at display 132 and/or input device 134) to interact with elements of graph 300 in GUI 301, such as by selecting, tapping, dragging, or otherwise directly interacting with elements of graph 300 to adjust autoregulation threshold offsets 310. For example, a clinician may provide user input at user interface 130 to adjust the blood pressure offset by which autoregulation threshold offsets 310 are offset from each other and from autoregulation threshold 308.

As shown in FIG. 3E, autoregulation threshold offsets 310 are offset from each other and from autoregulation threshold 308 by a specified blood pressure offset, such as 5 mmHg. A clinician may provide user input in the form of a drag gesture, such as at display 132, to select and drag autoregulation threshold offset 310B to a location in graph 300 associated with a particular blood pressure value in order to adjust the blood pressure offset associated with autoregulation threshold offsets 310. For example, the clinician may drag autoregulation threshold offset 310B to be closer to autoregulation threshold 308 in graph 300 in order to decrease the blood pressure offset associated with autoregulation threshold offsets 310 or may drag autoregulation threshold offset 310B to be further away from autoregulation threshold 308 in graph 300 in order to increase the blood pressure offset associated with autoregulation threshold offsets 310.

In the example of FIG. 3E, the clinician may draft autoregulation threshold offset 310B to be further away from autoregulation threshold 308 in graph 300 in order to increase the blood pressure offset associated with autoregulation threshold offsets 310 from 5 mm Hg to 10 mmHg. Processing circuitry 110 may therefore be configured to update graph 300 to update autoregulation threshold offsets 310 based on the updated blood pressure offset, such as to increase the blood pressure offset associated with autoregulation threshold offsets 310 from 5 mmHg to 10 mmHg.

In the example of FIG. 3F, GUI 301 includes a user interface element, such as text field 316, that enables a clinician to input the blood pressure offset associated with autoregulation threshold offsets 310. Processing circuitry 110 may be configured to receive, from user interface 130, an indication of the user input that corresponds to input of a blood pressure offset in text field 316 and may be configured to, in response, update graph 300 to update autoregulation threshold offsets 310 based on the inputted blood pressure offset. For example, if the clinician enters a blood pressure offset of 5 mmHg, processing circuitry 110 may be configured to, in response, update autoregulation threshold offsets 310 in graph 300 so that autoregulation threshold offsets 310 are offset by 5 mmHg from each other and from 308. Similarly, if the clinician enters a blood pressure offset of 10 mmHg, processing circuitry 110 may be configured to, in response, update autoregulation threshold offsets 310 in graph 300 so that autoregulation threshold offsets 310 are offset by 10 mmHg from each other and from 308.

In some examples, different types of surgeries may be associated with different autoregulation threshold offsets (e.g., different blood pressure offsets) and different autoregulation thresholds. For example, cardiovascular (CVOR) surgery may be associated with autoregulation threshold offsets and autoregulation thresholds that are different compared to non-cardiovascular surgery.

In the example of FIG. 3G, GUI 301 includes a user interface element, such as dropdown field 318, that enables a clinician to select the type of surgery that patient 101 is undergoing, such as to indicate whether patient 101 is undergoing cardiovascular surgery or non-cardiovascular surgery. Processing circuitry 110 may be configured to receive, from user interface 130, an indication of the user input that corresponds to selection of a type of surgery in dropdown field 318, and may be configured to, in response, update graph 300 with autoregulation threshold offsets 310 and/or autoregulation threshold 308 associated with the selected type of surgery.

In some examples, GUI 301 may be able to indicate both cerebral autoregulation status of patient 101 as well as non-cerebral (e.g., renal) autoregulation status of patient 101. The different types of autoregulation status may be associated with different autoregulation threshold offsets (e.g., different blood pressure offsets) and different autoregulation thresholds.

In the example of FIG. 3H, GUI 301 includes a user interface element, such as dropdown field 320, that enables a clinician to select the type of autoregulation status to be presented by graph 300 in autoregulation threshold offsets 310, such as to select the cerebral autoregulation status of patient 101 or to select the renal autoregulation status of patient 101. Processing circuitry 110 may be configured to receive, from user interface 130, an indication of the selected type of autoregulation status in dropdown field 320, and may be configured to, in response, update graph 300 with autoregulation threshold offsets 310 and/or autoregulation threshold 308 associated with the selected type of autoregulation status.

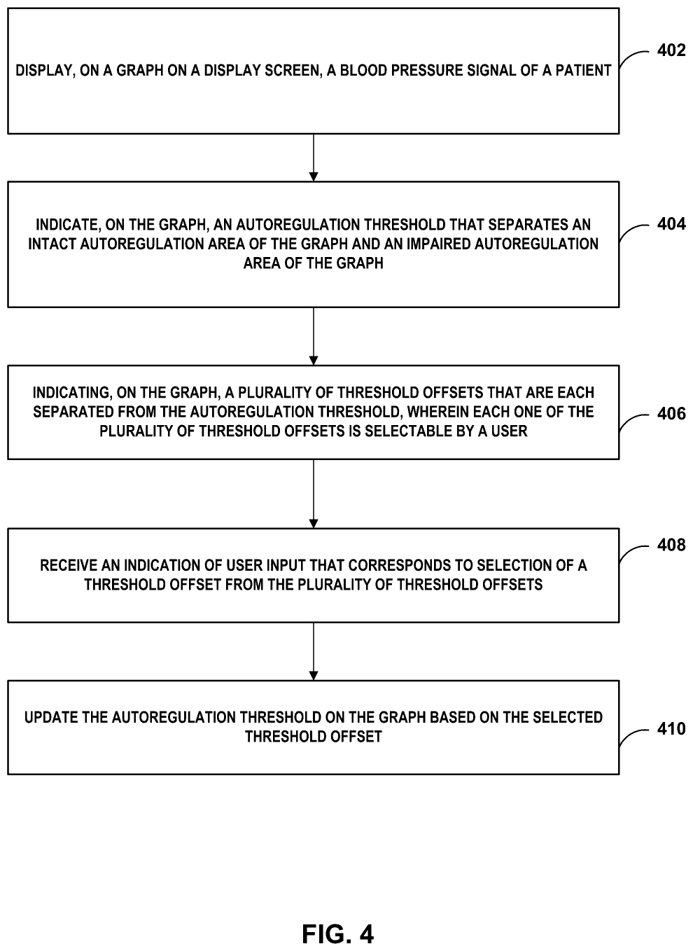

FIG. 4 is a flow diagram illustrating an example technique for presenting the autoregulation status of a patient. Although FIG. 4 is described with respect to processing circuitry 110 of autoregulation monitoring device 100 (FIGS. 1 and 2), in other examples, different processing circuitry, alone or in combination with processing circuitry 110, may perform any part of the technique of FIG. 4.

As shown in FIG. 4, processing circuitry 110 of autoregulation monitoring device 100 may display, on a graph on the display screen 132, a blood pressure signal of a patient 101 (402). Processing circuitry 110 may indicate, on the graph, an autoregulation threshold that separates an intact autoregulation area of the graph and an impaired autoregulation area of the graph (404). Processing circuitry 110 may indicate, on the graph, a plurality of threshold offsets that are each separated from the autoregulation threshold, wherein each one of the plurality of threshold offsets is selectable by a user (406). Processing circuitry 110 may receive an indication of user input that corresponds to selection of a threshold offset from the plurality of threshold offsets (408). Processing circuitry 110 may update the autoregulation threshold on the graph based on the selected threshold offset (410).

The techniques described in this disclosure, including those attributed to device 100, processing circuitry 110, control circuitry 122, sensing circuitries 140, 142, or various constituent components, may be implemented, at least in part, in hardware, software, firmware or any combination thereof. For example, various aspects of the techniques may be implemented within one or more processors, including one or more microprocessors, DSPs, ASICS, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

The following clauses include example subject matter described herein.

Clause 1. A method for displaying autoregulation status, comprising: displaying, on a graph on a display screen, a blood pressure signal of a patient; indicating, on the graph, an autoregulation threshold that separates an intact autoregulation area of the graph and an impaired autoregulation area of the graph; indicating, on the graph, a plurality of threshold offsets that are each separated from the autoregulation threshold, wherein each one of the plurality of threshold offsets is selectable by a user; receiving, by one or more processors, an indication of user input that corresponds to selection of a threshold offset from the plurality of threshold offsets; and updating, by the one or more processors, the autoregulation threshold on the graph based on the selected threshold offset.

Clause 2. The method of clause 1, further comprising: determining, by the one or more processors, a blood pressure offset, wherein the plurality of threshold offsets are offset from the autoregulation threshold and from each other by the blood pressure offset.

Clause 3. The method of clause 2, wherein the plurality of threshold offsets includes a first one or more threshold offsets that are above the autoregulation threshold and that are offset from each other and from the autoregulation threshold by the blood pressure offset, and a second one or more threshold offsets that are below the autoregulation threshold and that are offset from each other and from the threshold by the blood pressure offset.

Clause 4. The method of any of clauses 2 and 3, wherein the autoregulation threshold and the blood pressure offset are specific to the patient out of a plurality of patients.

Clause 5. The method of any of clauses 2-4, wherein the autoregulation threshold and the blood pressure offset are specific to a type of surgery that the patient is undergoing.

Clause 6. The method of any of clauses 2-5, wherein the autoregulation threshold and the blood pressure offset are specific to an autoregulation status of the patient being a non-cerebral autoregulation status of the patient.

Clause 7. The method of any of clauses 2-6, further comprising: receiving, by the one or more processors and from a computing system, indications of the autoregulation threshold and the blood pressure offset.

Clause 8. The method of any of clauses 2-7, wherein the blood pressure offset is a specified percentage of the autoregulation threshold.

Clause 9. The method of any of clauses 1-8, wherein a particular threshold offset out of the plurality of threshold offsets and the autoregulation threshold define an autoregulation threshold offset area in the graph between the particular threshold offset and the autoregulation threshold.

Clause 10. The method of clause 9, wherein the intact autoregulation area of the graph, the impaired autoregulation area of the graph, and the autoregulation threshold offset area in the graph are represented by different colors in the graph.

Clause 11. The method of any of clauses 1-10, wherein updating the autoregulation threshold on the graph based on the selected threshold offset further comprises: updating, by the one or more processors, a location of the autoregulation threshold on the graph to correspond to the selected threshold offset.

Clause 12. The method of any of clauses 1-11, wherein the user input comprises a drag gesture to drag the autoregulation threshold indicated on the graph to a location on the graph that corresponds to the selected threshold offset.

Clause 13. The method of any of clauses 2-12, further comprising: receiving, by the one or more processors, an indication of user input that corresponds to adjusting the blood pressure offset to an updated blood pressure offset; and in response to receiving the indication of user input that corresponds to adjusting the blood pressure offset to the updated blood pressure offset, updating, by the one or more processors, the threshold offsets in the graph based on the adjustment of the blood pressure offset to the updated blood pressure offset.

Clause 14. A device comprising: memory; and processing circuitry configured to: display, on a graph on the display screen, a blood pressure signal of a patient; indicate, on the graph, an autoregulation threshold that separates an intact autoregulation area of the graph and an impaired autoregulation area of the graph; indicate, on the graph, a plurality of threshold offsets that are each separated from the autoregulation threshold, wherein each one of the plurality of threshold offsets is selectable by a user; receive an indication of user input that corresponds to selection of a threshold offset from the plurality of threshold offsets; and update the autoregulation threshold on the graph based on the selected threshold offset.

Clause 15. The device of clause 14, wherein the processing circuitry is further configured to: determine a blood pressure offset, wherein the plurality of threshold offsets are offset from the autoregulation threshold and from each other by the blood pressure offset.

Clause 16. The device of clause 15, wherein the plurality of threshold offsets include a first one or more threshold offsets that are above the autoregulation threshold and that are offset from each other and from the autoregulation threshold by the blood pressure offset and a second one or more threshold offsets that are below the autoregulation threshold and that are offset from each other and from the autoregulation threshold by the blood pressure offset.

Clause 17. The device of any of clauses 15-16, wherein the autoregulation threshold and the blood pressure offset are specific to the patient out of a plurality of patients.

Clause 18. The device of any of clauses 15-17, wherein the autoregulation threshold and the blood pressure offset are specific to a type of surgery that the patient is undergoing.

Clause 19. The device of any of clauses 15-18 wherein the autoregulation threshold and the blood pressure offset are specific to an autoregulation status of the patient being a non-cerebral autoregulation status of the patient.

Clause 20. The device of any of clauses 15-19, wherein the processing circuitry is further configured to: receive, from a computing system, indications of the autoregulation threshold and the blood pressure offset.

Clause 21. The device of any of clauses 15-20, wherein the blood pressure offset is a specified percentage of the autoregulation threshold.

Clause 22. The device of any of clauses 15-21, wherein a particular threshold offset out of the plurality of threshold offsets and the autoregulation threshold define an autoregulation threshold offset area in the graph between the particular threshold offset and the autoregulation threshold.

Clause 23. The device of clause 22, wherein the intact autoregulation area of the graph, the impaired autoregulation area of the graph, and the autoregulation threshold offset area in the graph are represented by different colors in the graph.

Clause 24. The device of any of clauses 14-23, wherein to update the autoregulation threshold on the graph based on the selected threshold offset, the processing circuitry is further configured to: update a location of the autoregulation threshold on the graph to correspond to the selected threshold offset.

Clause 25. The device of any of clauses 14-24, wherein the user input comprises a drag gesture to drag the autoregulation threshold indicated on the graph to a location on the graph that corresponds to the selected threshold offset.

Clause 26. The device of any of clauses 15-25, wherein the processing circuitry is further configured to: receive an indication of user input that corresponds to adjusting the blood pressure offset to an updated blood pressure offset; and in response to receiving the indication of user input that corresponds to adjusting the blood pressure offset to the updated blood pressure offset, update the threshold offsets in the graph based on the adjustment of the blood pressure offset to the updated blood pressure offset.

Clause 27. A non-transitory computer readable medium comprising instructions that, when executed by processing circuitry, cause the processing circuitry to perform the method of any of clauses 1-13.

Various examples of the disclosure have been described. Any combination of the described systems, operations, or functions is contemplated. These and other examples are within the scope of the following claims.

What is claimed is:

1. A method for displaying autoregulation status, comprising:
displaying, on a graph on a display screen, a blood pressure signal of a patient;
indicating, on the graph, an autoregulation threshold that separates an intact autoregulation area of the graph and an impaired autoregulation area of the graph;
indicating, on the graph, a plurality of threshold offsets that are each separated from the autoregulation threshold, wherein each one of the plurality of threshold offsets is selectable by a user;
receiving, by one or more processors, an indication of user input that corresponds to selection of a threshold offset from the plurality of threshold offsets; and
updating, by the one or more processors, the autoregulation threshold on the graph based on the selected threshold offset.

2. The method of claim 1, further comprising:
determining, by the one or more processors, a blood pressure offset, wherein the plurality of threshold offsets are offset from the autoregulation threshold and from each other by the blood pressure offset.

3. The method of claim 2, wherein the plurality of threshold offsets includes a first one or more threshold offsets that are above the autoregulation threshold and that are offset from each other and from the autoregulation threshold by the blood pressure offset, and a second one or more threshold offsets that are below the autoregulation threshold and that are offset from each other and from the threshold by the blood pressure offset.

4. The method of any of claim 2, wherein the autoregulation threshold and the blood pressure offset are specific to the patient out of a plurality of patients.

5. The method of claim 2, wherein the autoregulation threshold and the blood pressure offset are specific to a type of surgery that the patient is undergoing.

6. The method of claim 2, wherein the autoregulation threshold and the blood pressure offset are specific to an autoregulation status of the patient being a non-cerebral autoregulation status of the patient.

7. The method of claim 2, further comprising:
receiving, by the one or more processors and from a computing system, indications of the autoregulation threshold and the blood pressure offset.

8. The method of any of claim 2, wherein the blood pressure offset is a specified percentage of the autoregulation threshold.

9. The method of any of claim 1, wherein a particular threshold offset out of the plurality of threshold offsets and the autoregulation threshold define an autoregulation threshold offset area in the graph between the particular threshold offset and the autoregulation threshold.

10. The method of claim 9, wherein the intact autoregulation area of the graph, the impaired autoregulation area of the graph, and the autoregulation threshold offset area in the graph are represented by different colors in the graph.

11. The method of claim 1, wherein updating the autoregulation threshold on the graph based on the selected threshold offset further comprises:
updating, by the one or more processors, a location of the autoregulation threshold on the graph to correspond to the selected threshold offset.

12. The method of claim 1, wherein the user input comprises a drag gesture to drag the autoregulation threshold indicated on the graph to a location on the graph that corresponds to the selected threshold offset.

13. The method of claim 2, further comprising:
receiving, by the one or more processors, an indication of user input that corresponds to adjusting the blood pressure offset to an updated blood pressure offset; and
in response to receiving the indication of user input that corresponds to adjusting the blood pressure offset to the updated blood pressure offset, updating, by the one or more processors, the threshold offsets in the graph based on the adjustment of the blood pressure offset to the updated blood pressure offset.

14. A device comprising:
memory; and
processing circuitry configured to:
display, on a graph on a display screen, a blood pressure signal of a patient;
indicate an autoregulation threshold that separates an intact autoregulation area of the graph and an impaired autoregulation area of the graph;
indicate a plurality of threshold offsets that are each separated from the autoregulation threshold, wherein each one of the plurality of threshold offsets is selectable by a user;
receive an indication of user input that corresponds to selection of a threshold offset from the plurality of threshold offsets; and
update the autoregulation threshold on the graph based on the selected threshold offset.

15. A non-transitory computer readable medium comprising instructions that, when executed by processing circuitry, cause the processing circuitry to:
display, on a graph on a display screen, a blood pressure signal of a patient;
indicate an autoregulation threshold that separates an intact autoregulation area of the graph and an impaired autoregulation area of the graph;
indicate a plurality of threshold offsets that are each separated from the autoregulation threshold, wherein each one of the plurality of threshold offsets is selectable by a user;
receive an indication of user input that corresponds to selection of a threshold offset from the plurality of threshold offsets; and
update the autoregulation threshold on the graph based on the selected threshold offset.
